(19) United States
(12) Patent Application Publication
(10) Pub. No.: US 2006/0127326 A1
(43) Pub. Date: Jun. 15, 2006

(54) Oral hygiene product for human and animal use with a melatonin base as an active product

(76) Inventors: Antonio Cutando Soriano, Armilla (GD); Dario Acuna Castroviejo, Granada (ES); Gerardo Gomez Moreno, Cajar (GD); Carlos Arana Molina, Granada (ES)

Correspondence Address: DAVIDSON, DAVIDSON & KAPPEL, LLC;14th Floor, 485 Seventh Avenue, New York, NY, 10018, US

(21) Appl. No.: 10/722,356

(22) Filed: Nov. 25, 2003

(30) Foreign Application Priority Data
Dec. 9, 2002 (ES) 200202810

(51) Int. Cl. A61K 8/49

(52) U.S. Cl. 424/49

(57) ABSTRACT

An oral hygiene product for human and animal use with a melatonin base as an active product is provided to be used as tooth paste, mouthwashes or animal feeds. The product includes a hydrophilic excipient, a hydrophobic excipient, and melatonin in a proportion of 0.1% to 5%.

### OBJECT OF THE INVENTION

This descriptive report refers to an Invention Patent request for an oral hygiene product for human and animal use with a melatonin base as an active product, to be applied on humans and animals, containing melatonin, using the anti-oxidant and immuno-stimulating activity of this compound.

When it is to be used on humans it is constituted as a toothpaste or mouthwash, whereas when it is to be used as a product for animal use the melatonin effects are best obtained by including this compound in food pellets.

### SCOPE OF THE INVENTION

This invention is to be used in industries manufacturing human and animal oral hygiene products.

### ANTECEDENTS OF THE INVENTION

All physiological processes depend on the capacity of cells to receive nutrients and eliminate waste products from the extracellular compartment and during these processes free radicals are generated. Free radical production significantly increases if there is any illness or lesion.

Free radicals formed in this way are classified into two groups, i.e.:
Oxygen free radicals and
Nitrogen free radicals.

The former, constituted as oxygen free radicals are derived from the incomplete reduction of oxygen with the superoxide anion and the highly toxic hydroxil radical.

Among the latter, more specifically the nitrogen free radicals, are nitric oxide (NO) and the peroxynitrites, which are very toxic and derived from the reaction between nitric oxide and the superoxide anion.

For its part, nitric oxide is produced by nitric oxide sintase (NOS), generating very large quantities of nitric oxide.

Under these conditions, levels of oxygen and nitrogen free radicals are very high, inducing damage and cell death and also creating and obtaining an increase in the production of free radicals that generate an increase in the rigidity of cell membranes and connective tissue due to crossed links. This causes a decrease in blood supply to organs and tissues with the consequential decrease in tissue perfusion. To counteract the accumulation of cytoxic bioproducts derived from the oxidative reactions necessary for life, living organisms have developed detoxification and DNA repair processes and natural anti-oxidants such as free radical purifiers, enzymes and protein decomposition.

Intercellular purification activity includes the production (probably under genetic control) of various anti-oxidants in response to oxidative reactions. In these systems it is noted that they are sufficient to give cellular protection, but do not produce toxic effects by its own anti-oxidant activity.

Another important consideration is age, given that the degenerative processes associated with age are in part a result of non-repaired damage to macromolecules, produced by free radicals. 2-5% of inhaled oxygen is converted into toxic oxygen radicals, meaning that when an individual reaches 70 years old he has produced over 700 kg of oxygen radicals. Although these oxygen radicals are the basis of the ageing process, they are also used by the cell for physiological processes such as in the case of activated fagocytes producing O2 to destroy bacteria or such as chemical mediators (activation of the transcription factor (MF-kB).

The anti-oxidant systems in the organism must allow these necessary functions of the oxygen radicals, but at the same time have to avoid their toxic effects.

Production of radicals is counteracted by an endogenous anti-oxidant that makes use of several endogenous and exogenous compounds, among which are SOD, catalase, glutation and GSH-PX plus vitamins A, C and E.

Under oral illness and ageing conditions, production of free radicals frequently exceeds the capacity to neutralise them and as such, many radicals are not neutralised and maintain a persistent bombardment of biologically important molecules. These are at least in part responsible for damage in these processes.

Melatonin is very important component in the anti-oxidant system of the organism and can change the redox status of the cell.

When melatonin detoxifies OH it becomes an indolyl radical cation with a very low toxicity level and this radical now purifies the superoxide anion and becomes a N-acetyl-N-formyl-5-metoxykinurenamine.

This series of reactions make melatonin an ideal purifier given that one molecule of melatonin purifies two free radical molecules and to do this, melatonin does not require a specific receptor acting directly on the cytosol and cell membrane. This means that it does not exclude its action via a nuclear receptor that genomically modulates some anti-oxidant enzymes such as poly (ADP ribose) polymerase, to repair DNA, peroxidase and reductase glutation, iNOS and others.

Melatonin actions deeply affect homeostasis, metabolism, the immune system, connective tissue maintenance and structural and muscular component maintenance. The last two, i.e. structural and muscular components are responsible for elasticity and rigidity. Melatonin directly regulates, via the calmodulin, tubuline synthesis forming microtubules, the structure that forms the cell cytoskeleton and as such the elasticity and/or cell rigidity.

Under normal conditions, a third of the melatonin circulating in the blood is excreted via saliva through the mouth. There is data showing that this saliva excretion of melatonin maintains suitable levels of the hormone protecting local areas of damaged mouth tissue that are continually being received as a result or consequence of the mouth's physiological functions. As a result it means that a supply of exogenous melatonin to the mouth will be very useful in preventing oxidative damage and stimulating a local immune response.

Furthermore, if it is considered that levels of melatonin in the blood decrease with age, the continuous supply of melatonin to the mouth in the form of toothpaste and/or mouthwash, may prevent the appearance of oral illnesses. The same is true for animals where oral care will improve if an extra supply of melatonin is given in the feed.

The destructive effects produced on oral tissues in living beings by pathologies, such as periodontopathies among others, or as a result of surgical interventions in the oral cavity, are significant and affect, via the inflammatory process, both the mucose such as bone tissue and other neighbouring tissues.

In these destructive processes in which there are different kinds of germs, oxidative stress, stemming from the said inflammatory process, plays a vital role. Here a large number of free radicals are released causing the cell destruction.

Medical treatments aimed at resolving these pathologies have concentrated on controlling the infection with antimicrobiotic drugs and controlling the inflammatory process concentrates on using generally non steroid anti-inflammatory drugs.

The idea of fighting free radicals produced in the inflammatory response decreasing cell destruction and allowing a better regeneration of affected tissue, matches the attempt the organism already makes by using certain orally produced anti-oxidants such as ascorbic acid, albumin and uric acid.

To do this, it has been proposed that vitamin A, E, CoQ among others are used as external and systemic anti-oxidants.

Both the anti-oxidant capacity and the results obtained are much lower than the capacity of melatonin and its derivatives. Currently melatonin, a natural substance produced by the organism up to a certain age in response to pathologies like this, has demonstrated a considerably higher power than those substances mentioned previously, entering the cell, even reaching its nucleus and protecting the free radicals produced during the inflammatory process thereby averting its destruction.

The applicant is aware of the current existence of Invention Patents WO 00/67708, U.S. Pat. No. 5,665,332, DE 196 15 820 and GB 990.082 referring to tooth pastes containing hormones.

Finally, invention patents are also known referring to pharmaceutical compounds administered externally or orally that include melatonin and that can be applied to animals in some cases identified by numbers WO 96/08490, WO 92/06955, WO 95/26713 and WO 92/02207.

It is reiterated that all these aforementioned documents are part of the state of technology previous to the invention described in this document. However they can in no way be considered to affect in any way the activity of this invention nor the novelty of the same.

### DESCRIPTION OF THE INVENTION

The oral hygiene product for human and animal use with a melatonin base as an active product is based on the use of melatonin which is a natural hormone produced by all living organisms form single cell beings to man and including plants.

Consequently melatonin is a natural not a synthetic product. However, melatonin in a pure form can be acquired from current suppliers of chemical products from where the product was obtained for research.

As previously mentioned, different research has demonstrated that it is an excellent natural anti-oxidant with immuno-modulatory actions. Based on this, this invention uses the properties of melatonin to use it as an oral protector. For this melatonin is introduced into toothpastes and into mouth washes. It will also be used for the same reasons on animals by including it as a part of animal feeds.

### SPECIAL ACTIONS OF THE INVENTION

The oral hygiene product for human and animal use with a melatonin base as an active product and in particular in preparing tooth pastes uses both hydrophile and hydrophibe bases or excipients that can be used in tooth pastes and/or mouthwash in the master formulation and in industrial preparation. Therefore melatonin will be used in a proportion of 0.1% to 5% in the said tooth pastes and/or mouthwashes.

For the preparation of animal feeds, melatonin will be added in a proportion of 0.1% to 5% in the said feeds.

The melatonin can be degraded by auto-oxidation or action by microbiotic enzymes to avoid auto-oxidation of the melatonin. Each of the previously mentioned preparations will include the most suitable anti-oxidant for each case.

The anti-oxidant will be active in low concentration and in a broad pH range, soluble in the medium used and colourless as far as possible, thermostable and of course non toxic non irritant and non volatile.

Among the most commonly used in formulation are sodium sulphite and bisulphite, ascorbic acid, hydroquinone, nipagin, vitamin E, vitamin A, etc.

Its effectiveness can be increased with the use of synergic substances such as citric and tartaric acids, as well as EDTA.

Preservative must be added to avoid degradation by microbiotic contamination. The preservative used must not be toxic, be chemically stable and compatible with melatonin. Among others benzoic acid, salicilic acid, sorbic acid and essential oils will be used.

The specific formula of this invention is as follows.

Melatonin:

N-acetyl-5-metoxitripamine

1. An oral hygiene product for human and animal use with a melatonin base as an active product to be used a tooth pastes, mouthwashes or animal feeds, comprising hydrophilic excipient, a hydrophobic excipient and melatonin in a proportion of 0.1% to 5%.

2. The oral hygiene product according to claim 1, further comprising a colourless, thermostable, non toxic, non volatile, non irritating an active auto-oxidant.

3. The oral hygiene product according to claim 1, further comprising a preservative.

4. The oral hygiene product according to claim 1, wherein the melatonin is
N-acetyl-5-metoxitripamine

5. The oral hygiene product according to claim 1, further comprising an active auto-oxidant selected from the group consisting of sodium sulphite, sodium bisulfate, ascorbic acid, hyroquinone, nipagin, vitamin E, and vitamin A.

6. The oral hygiene product according to claim 1, further comprising a preservative selected from the group consisting of benzoic acid, salicilic acid, sorbic acid, and essential oils.
